(19) United States
(12) Patent Application Publication
(10) Pub. No.: US 2017/0145763 A1
(43) Pub. Date: May 25, 2017

(54) Drilling Rig and Method of Operating It

(71) Applicant: Endress + Hauser Messtechnik GmbH + Co. KG, Weil am Rhein (DE)

(72) Inventors: Ingo Knudsen, Rieseby (DE); Rob Vermeulen, Greenwood, IN (US)

(21) Appl. No.: 14/331,284

(22) Filed: Jul. 15, 2014

(51) Int. Cl. E21B 21/08; E21B 47/06; E21B 47/04; G01N 33/28; E21B 21/00; G01F 23/18; G01N 9/26; E21B 7/12; E21B 47/00

(52) U.S. Cl. CPC E21B 21/08; E21B 7/12; E21B 47/06; E21B 47/042; E21B 47/0001; E21B 21/001; G01F 23/18; G01N 9/26; G01N 33/2823; E21B 21/065

(57) ABSTRACT

A drilling rig which provides information, which may be used to detect abnormal drilling mud backflow, comprising: a drill string for drilling a borehole comprising a drill bit at its end, structure for pumping drill mud down the borehole inside the drill string through the drill bit, and up the borehole on an outside of the drill string, a riser surrounding the drill string above the borehole, an annulus enclosed between the riser and the drill string forming a passageway for drill mud rising from the bottom of the borehole on the outside of the drill string structure for measuring a level of the drill mud inside the annulus inside the riser above the borehole, comprising: a first pressure measurement device for measuring a first pressure prevailing inside the annulus at a first height and a second pressure measurement device for measuring a second pressure prevailing inside the annulus at a second height, structure for determining a density of the drill mud, based on a ratio of a pressure difference between the first pressure and the second pressure and a product of a height difference between the first and the second height and the constant of gravity, and means for determining the level, based on a ratio of the first pressure and a product of the density and the constant of gravity and/or based on a ratio of the second pressure and a product of the density and the constant of gravity.

### BACKGROUND AND SUMMARY OF THE INVENTION

The present invention concerns a drilling rig for drilling a well, in particular an oil or gas well, comprising, a drill string for drilling a borehole comprising a drill bit at its end, means for pumping drill mud down the borehole inside the drill string through the drill bit, and up the borehole on an outside of the drill string, a riser surrounding the drill string above the borehole, and an annulus enclosed between the riser and the drill string forming a passageway for drill mud rising from the bottom of the borehole on the outside of the drill string.

Drilling rigs are e.g. used in the oil and gas industry for drilling wells.

Drilling rigs generally comprise a drill string for drilling a borehole comprising a drill bit at its end, means for pumping drill mud down the borehole inside the drill string through the drill bit, and up the borehole on an outside of the drill string, a riser surrounding the drill string above the borehole, and an annulus enclosed between the riser and the drill string forming a passageway for drill mud rising from the bottom of the borehole on the outside of the drill string.

The drill mud circulated through the borehole serves the purpose of stabilizing the sidewalls of the borehole and of transporting the drill cuttings from the bottom of the borehole up to the surface. During normal operation, drill mud having a density suitable for this purpose is pumped down the borehole with a constant pumping pressure.

During drilling operation it is possible, that the drill bit hits formations enclosing a pressurized gas or fluid. When gases or fluids under high pressure enter the borehole, the pressure increase inside the borehole can cause a blow out at the surface.

It is described in U.S. Pat. No. 4,297,880 to detect intrusions of materials, having a density different form the density of the drill mud, into the borehole, by measuring a pressure difference between a pressure inside and outside the drill string near the drill bit.

US 2009/0205822 A1 describes a method of monitoring the density of the drill mud. To this extend, first and second pressure measurement means are foreseen for measuring the pressure of the drill mud returning to the surface at two different heights along the well bore. Changes of the density of the drill mud, e.g. caused by gas intruding into the borehole, are determined based on corresponding changes of the pressure differences between simultaneously measured first and second pressures in relation to the product of the height difference between the two heights and the constant of gravity.

US 2009/0205822 A1 describes to detect changes of the drill mud density, e.g. caused by a gas influx, and to take corrective actions, e.g. to seal of the well, in response to determined density changes. According to US 2009/0205822 A1 the pressure measurement means are preferably located near the drill bit, but could also be foreseen along the length of the drill string or may be placed in a stationary location, e.g. a cement shoe or a casing. Moreover, it is mentioned to utilize the system described in US 2009/0205822 A1 along fluid conduits such as flow lines, risers and pipes.

Obviously, installing pressure measurement means at the preferred location near the drill bit, in order to detect changes of the drill mud density at an early stage, requires for the pressure measurement means to be lowered down the bore hole, and for the measurement results to be transferred up to the surface.

In addition, problems causing an abnormal drilling mud backflow, which do not affect the density of the drill mud within the height range covered by the pressure measurement means cannot be detected by this method.

It is an object of the invention to provide a drilling rig and a method of operating said drilling rig, providing additional information, which may be used to detect abnormal drilling mud backflow.

To this extent, the invention comprises a rig for drilling a well, in particular an oil or gas well, comprising,
a drill string for drilling a borehole comprising a drill bit at its end,
means for pumping drill mud down the borehole inside the drill string through the drill bit, and up the borehole on an outside of the drill string,
a riser surrounding the drill string above the borehole,
an annulus enclosed between the riser and the drill string forming a passageway for drill mud rising from the bottom of the borehole on the outside of the drill string,
means for measuring a level of the drill mud inside the annulus inside the riser above the borehole, comprising:
   a first pressure measurement device for measuring a first pressure prevailing inside the annulus at a first height and a second pressure measurement device for measuring a second pressure prevailing inside the annulus at a second height,
   means for determining a density of the drill mud based on a ratio of a pressure difference between the first pressure and the second pressure and a product of a height difference between the first and the second height and the constant of gravity, and
   means for determining the level based on a ratio of the a first pressure and a product of the density and the constant of gravity and/or based on a ratio of the second pressure and a product of the density and the constant of gravity.

According to a first preferred embodiment, the first and the second pressure measurement device are located below a mud return line, allowing for drill mud rising inside the annulus to flow out of the annulus.

According to a second preferred embodiment the rig comprises a monitoring unit for monitoring the level and detecting an abnormal mud backflow, in case the monitored level changes by more than a predetermined threshold.

According to a refinement of the second preferred embodiment, the monitoring unit monitors the density and detects an abnormal mud backflow, in case the monitored density changes by more than a predetermined threshold.

The invention further comprises a drilling rig according to the second preferred embodiment or the refinement thereof, wherein
the monitoring unit comprises an internal clock, and
the monitored levels and/or the monitored densities are monitored as a function of time.

The invention further comprises a method of operating a drilling rig according to the invention, comprising the steps of:
monitoring the level (L) determined by the means for measuring the level (L), and
detecting an abnormal drill mud backflow in case the monitored level (L) changes by more than a predetermined threshold.

The invention further comprises a refinement of the method according to the invention, comprising the steps of:
detecting an abnormal drill mud backflow indicative of an onset of a potential blow out, in case the monitored level rises by more than a predetermined threshold, and/or
detecting an abnormal drill mud backflow indicative of a loss of drill mud, in case the monitored level drops by more than a predetermined threshold.

The invention further comprises a refinement of the method according to the invention or the refinement thereof, comprising the steps of:
monitoring the densities determined by the means for determining the density, and
detecting an abnormal drill mud backflow in case the monitored density changes by more than a predetermined threshold.

A further refinement of the last mentioned method further comprises the step of detecting an abnormal drill mud backflow indicative of an onset of a potential blow out, in case the monitored density drops by more than a predetermined threshold.

It is an advantage of the invention, that the level of the drill mud inside the riser provides information, which may be used to detect abnormal drilling mud backflow. By monitoring the level, abnormal drilling mud backflows indicative of an onset of a threatening blow out or indicative of a loss of drill mud can be easily detected.

According to the invention, the required pressure measurement devices and the measurement electronic are located above the borehole. This has the advantage, that they can be easily installed, connected up and are easily accessible whenever necessary. It is not necessary to lower measurement devices down the bore hole, or to transfer measurement results up to the surface.

The invention and further advantages are explained in more detail using the FIGURE of the drawing, in which one exemplary embodiment is shown.

### BRIEF DESCRIPTION OF THE DRAWING

FIG. 1 shows: a drilling rig.

### DETAILED DESCRIPTION OF THE DRAWINGS

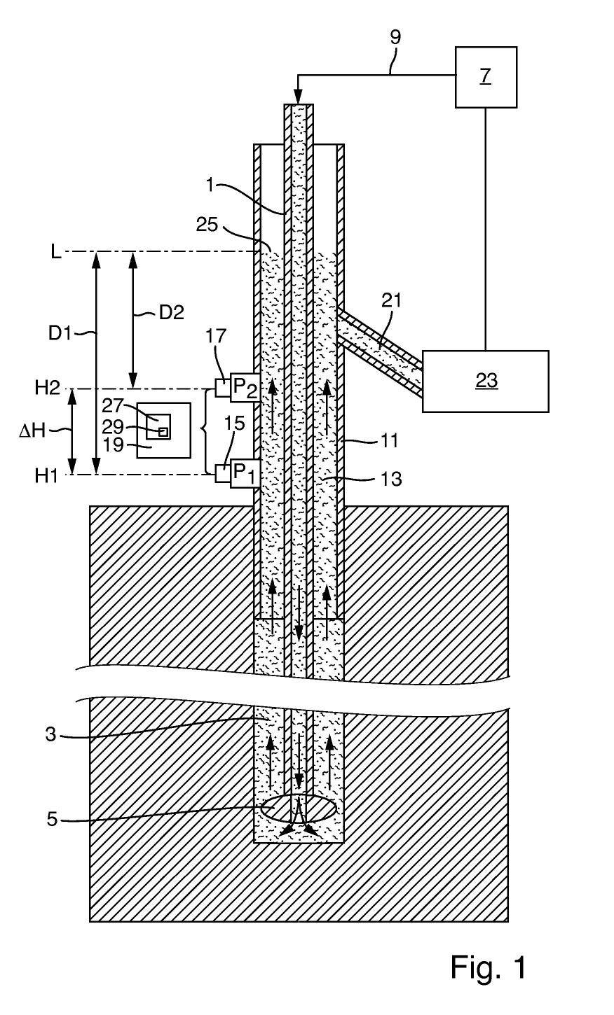

FIG. 1 shows a schematic diagram of drilling rig for drilling a well, in particular an oil or gas well. The rig comprises a drill string 1 for drilling a borehole 3 and a drill bit 5 located at the end of the drill string 1.

The drill string 1 is hollow, and means 7 are foreseen for pumping drill mud down the drill string 1 through the drill bit 5 into the borehole 3. These means 7 comprise e.g. a pump for pumping drill mud through a supply line 9 connected to the drill string 1. From the bottom of the borehole 3, the drill mud will then return up the borehole 3 on an outside of the drill string 1. Above the borehole 3 a riser 11 is foreseen, which encloses a section of the drill string 1, which is located above the borehole 3. An annulus 13 enclosed between the riser 11 and the drill string 1 forms a passageway for drill mud returning from the bottom of the borehole 3 on the outside of the drill string 1, through which the returning drill mud rises up.

According to the invention, the rig comprises measurement means for measuring a level L of the drill mud inside the annulus 13 inside the riser 11 above the borehole 3.

These means comprise a first pressure measurement device 15 for measuring a first pressure p₁, prevailing inside the annulus 13 at a first height H₁ above the borehole 3 and a second pressure measurement device 17 for measuring a second pressure p₂ prevailing inside the annulus 13 at a second height H₂ above the borehole 3. The pressure measurement devices 15, 17 comprise e.g. a first pressure sensor, installed in an opening foreseen in the riser 11 at the first height H1, and a second pressure sensor, installed in an opening foreseen in the riser 11 at the second height H2.

In addition the means for determining the level L of the drill mud inside the riser 11 comprise a measurement electronic 19 connected to the first and the second pressure measurement device 15, 17

Both pressure measurement devices 15, 17 are located below a mud return line 21, allowing for drill mud rising inside the annulus 13 to flow out of the annulus 13. The drill mud flowing out of the annulus 13 is supplied to a mud recirculation system 23 designed to separate the drill cutting contained in the drill mud from the drill mud and to recondition the drill mud, which can then be resupplied to the drill string 1 via the supply line 9.

During operation of the rig, the first and the second pressure p₁, p₂ are measured by the first and the second pressure measurement device 15, 17, and the measurement electronic 19 determines the density [symbol] of the drill mud based on a pressure difference Δp=p₁−p₂ between the first pressure p₁ and the second pressure p₂ and a product of a height difference ΔH=H2−H1 between the first height H1 and the second height H2 and the constant of gravity g. The density [symbol] is given by:

$$ρ = \frac{{p}_{1} - {p}_{2}}{g Δ H}$$

Based on the determined density [symbol] and the first or the second pressure p₁, p₂ a density compensated the level L of the drill mud inside the annulus 13 of the riser 11 is determined. To this extend, the measurement electronic 19 determines the level L based on the first pressure p₁ and a product of the density [symbol] and the constant of gravity g and/or based on the second pressure p₂ and a product of the density [symbol] and the constant of gravity g. In either case, the level L corresponds to the position of a surface 25 of the drill mud inside the annulus 13 which can e.g. be determined as a distance D1, D2 between the surface 25 and the height H1, H2 at which the respective pressure p₁, p₂ is measured.

The level L corresponding to the position of a surface 25 of the drill mud inside the annulus 13 can be determined as a distance D1 between the surface 25 and the first height H1 at which the first pressure p₁ is measured, given by:

$$D 1 = \frac{{p}_{1}}{ρ * g}$$

and/or it can be determined as a distance D2 between the surface 25 and the second height H2 at which the second pressure p₂ is measured, given by:

$$D 2 = \frac{{p}_{2}}{ρ * g}$$

During normal operation of the rig, drill mud having a suitable density is supplied to the rig, and pumped down the borehole inside the drill string 1 with a constant pumping pressure. In consequence the drill mud returning to the surface will rise up to a level L, which remains fairly constant during normal operation, and will have a fairly constant density [symbol] .

Thus during operation of the rig, the level L of the drill mud inside the riser 11 is monitored based on the level measurements performed by the level measurement means provided on the rig. Monitoring is performed by a monitoring unit 27, which can be an integral part of the measurement electronic 19, or a separate unit connected to the measurement electronic 19. The monitoring unit 27 is designed to detect an abnormal drill mud backflow in case the monitored level L changes by more than a predetermined threshold.

Preferably the monitoring unit 27 is designed to not only monitor the determined levels L, but also the determined densities [symbol] , and to detect an abnormal drill mud backflow in case the monitored density [symbol] changes by more than a predetermined threshold.

The monitoring unit 27 preferably comprises an internal clock 29, and the monitored levels L and the monitored densities are monitored as a function of time.

If a gas or a liquid under high pressure enters the borehole 3, the pressure increase inside the borehole 3 will exert an additional force onto the drill mud, pushing the drill mud up the borehole 3 on the outside of the drill string 1. The additional pressure exerted by the gas or liquid influx, will thus cause the level L of the drill mud inside the riser 11 to rise. In consequence an abnormal drill mud backflow indicative of the onset of a potential blow out will be detected by the monitoring unit 27, in case the monitored level L rises by more than a predetermined threshold.

The increase of the level L as well as the speed at which it increases depends on the pressure exerted by the influx in relation to the counter pressure exerted by the drill mud, in particular the overhead pressure exerted by the mud column above the location of the influx, the type of influx and the consistency of the drill mud.

In most cases, higher pressures exerted by the influx will cause the level L to increase higher and faster. Depending on the situation at the location of the influx, in particular the pressures acting there, the type of influx and the consistency of the drill mud, it is very well possible, that the pressures exerted by the influx cause the level L of the drill mud to increase inside the riser 11 well before drill mud containing the gas or liquid will return to the surface.

Gas or liquid contained in the drill mud reduces the density [symbol] of the drill mud. Thus gas or liquid contained in the drill mud reaching the section of the riser 11 equipped with the pressure measurement devices 15, 17 can be detected based on the monitored density [symbol] . In consequence an abnormal drill mud backflow indicative of the onset of a potential blow out will be detected by the monitoring unit 27, in case the monitored density [symbol] drops by more than a predetermined threshold.

In all cases where the level L inside the riser 11 rises before the drill mud containing the gas or liquid returns to the surface, the level increase caused by the influx, will be detected before a change in density [symbol] caused by the influx will be detected. In these cases, an abnormal mud backflow, indicative of the onset of a threatening blow out, will be detected by the rig according to the invention earlier, than would be possible based on the density measurements.

In all other cases, monitoring the level L and monitoring the density [symbol] constitute two different methods of detecting abnormal mud backflow indicative of the onset of a threatening blow out, which are preferably applied in parallel, in order to enhance the probability of an early detection of the onset of a threatening blow out.

In case an abnormal drill mud backflow, indicative of an onset of a threatening blow out is detected corresponding safety measures, e.g. the activation of blow out preventers foreseen on the rig, are applied.

If the drill bit 5 hits a cavity, drill mud supplied through the drill string 1 to the bottom of the borehole will enter the cavity. In this case, the level L inside the riser 11 will drop due to the amount of drill mud lost into the cavity. Correspondingly, the monitoring unit 27 will detect an abnormal drill mud backflow indicative of a loss of drill mud, in case the monitored level L drops by more than a predetermined threshold. In case an abnormal drill mud backflow, indicative of a loss of drill mud is detected a corresponding warning or an alarm can be issued, and suitable safety measures, e.g. measures for sealing the borehole walls, can be applied.
1 drill string
3 borehole
5 drill bit
7 means for pumping drill mud
9 supply line
11 riser
13 annulus
15 first pressure measurement device
17 second pressure measurement device
19 measurement electronic
21 return line
23 mud recirculation system
25 surface of the drill mud
27 internal clock

1-9. (canceled)

10. A drilling rig for drilling a well, in particular an oil or gas well, comprising:
a drill string for drilling a borehole, comprising a drill bit at one end, means for pumping drill mud down the borehole inside said drill string through said drill bit, and up the borehole on an outside of said drill string;
a riser surrounding said drill string above the borehole;
an annulus enclosed between said riser and said drill string forming a passageway for drill mud rising from the bottom of the borehole on the outside of said drill string;
means for measuring a level of the drill mud inside said annulus inside said riser above the borehole, comprising:
a first pressure measurement device for measuring a first pressure prevailing inside said annulus at a first height and a second pressure measurement device for measuring a second pressure prevailing inside the annulus at a second height;
means for determining a density of the drill mud based on a ratio of a pressure difference between said first pressure and said second pressure and a product of a height difference between said first and said second height and the constant of gravity; and
means for determining said level based on a ratio of said first pressure and a product of the density and the constant of gravity and/or based on a ratio of said second pressure and a product of the density and the constant of gravity.

11. The drilling rig according to claim 10, wherein:
said first and said second pressure measurement device are located below a mud return line, allowing for drill mud rising inside said annulus to flow out of said annulus.

12. The drilling rig according to claim 10, further comprising:
a monitoring unit for monitoring the level and detecting an abnormal mud backflow, in case the monitored level changes by more than a predetermined threshold.

13. The drilling rig according to claim 12, wherein:
said monitoring unit monitors the density and detects an abnormal mud backflow, in case the monitored density changes by more than a predetermined threshold.

14. The drilling rig according to claim 12, wherein:
said monitoring unit comprises an internal clock; and
said monitored levels and/or the monitored densities are monitored as a function of time.

15. A method of operating a drilling rig, comprising the steps of:
monitoring the level determined by means for measuring the level; and
detecting an abnormal drill mud backflow in case the monitored level changes by more than a predetermined threshold.

16. The method according to claim 15, comprising the steps of:
detecting an abnormal drill mud backflow indicative of an onset of a potential blow out, in case a monitored level rises by more than a predetermined threshold; and/or
detecting an abnormal drill mud backflow indicative of a loss of drill mud, in case the monitored level drops by more than a predetermined threshold.

17. The method according to claim 15, comprising the steps of:
monitoring the densities determined by the means for determining the density; and
detecting an abnormal drill mud backflow in case the monitored density changes by more than a predetermined threshold.

18. The method according to claim 17, comprising the step of:
detecting an abnormal drill mud backflow indicative of an onset of a potential blow out, in case the monitored density drops by more than a predetermined threshold.
